(19) United States
(12) Patent Application Publication
(10) Pub. No.: US 2024/0307044 A1
(43) Pub. Date: Sep. 19, 2024

(54) MEDICAL DELIVERY SYSTEMS, DEVICES, AND RELATED METHODS OF USE

(71) Applicants: Boston Scientific Scimed, Inc., Maple Grove, MN (US); Boston Scientific Medical Device Limited, Galway (IE)

(72) Inventors: Nachiket GOLE, Pune (IN); Deepak Kumar SHARMA, Muzaffarnagar (IN); Rajivkumar SINGH, Thane (IN); Sharath Kumar G, Kanakapura (IN); Kevin L. BAGLEY, Natick, MA (US)

(73) Assignees: Boston Scientific Scimed, Inc., Maple Grove, MN (US); Boston Scientific Medical Device Limited, Galway (IE)

(21) Appl. No.: 18/599,341

(22) Filed: Mar. 8, 2024

Related U.S. Application Data
(60) Provisional application No. 63/490,008, filed on Mar. 14, 2023.

(51) Int. Cl. A61B 17/00; A61B 1/00; A61B 1/015; A61B 17/12

(52) U.S. Cl. CPC A61B 17/00491; A61B 1/00089; A61B 1/00101; A61B 1/015; A61B 2017/00818; A61B 2017/12004

(57) ABSTRACT

A delivery device comprises a tube having a proximal end, a distal end, and a lumen extending from the proximal to the distal end. The proximal end is mountable to a distal tip of a medical device via the lumen. The tube includes an inner wall defining the lumen, an outer wall coupled to the inner wall at the proximal and distal ends, a cavity formed from the coupled inner and outer walls and configured to hold a deliverable substance, and a plurality of openings connected to the cavity and positioned on a distal-facing surface of the distal end. At least a portion of the inner wall or the outer wall is a flexible wall that transitions between a first and a second configuration to cause delivery of the deliverable substance from the cavity to a treatment site through the openings when a force is applied to the flexible wall.

### CROSS-REFERENCE TO RELATED APPLICATIONS

This application claims priority to U.S. Provisional Application No. 63/490,008, filed on Mar. 14, 2023, which is incorporated by reference herein in its entirety.

### TECHNICAL FIELD

The disclosure relates generally to medical delivery systems, devices, and related methods of use. More specifically, aspects of the disclosure pertain to delivery devices of various configurations for mounting to medical devices to deliver substances, such as adhesive, to a treatment site.

### BACKGROUND

Bleeding in the gastrointestinal (GI) tract is often challenging to control, as it can be difficult to access a site of the bleeding. One exemplary treatment method to control bleeding in the GI tract includes application of a hemostatic device, such as a patch or mesh, to the site. For example, the patch or mesh may be deployed to the site using an endoscope, and a liquid or semi-liquid adhesive may be applied to secure the patch or the mesh to the site until at least clots are formed to stop the bleeding. Currently, the adhesive may be delivered through an injection needle that is delivered distally through a working channel of the endoscope to reach the site. However, the injection needle is limited to delivering the adhesive at a single point or location, and occupies a working channel of the endoscope. Therefore, the injection needle may have to be repeatedly repositioned and actuated to deliver the adhesive to multiple points or location throughout the site to help ensure the patch or the mesh is sufficiently secured to the site, which may increase an overall procedure time.

### SUMMARY

A delivery device may include a tube having a proximal end, a distal end, and a lumen extending from the proximal end to the distal end. The proximal end of the tube may be mountable to a distal tip of a medical device via the lumen. The tube may include an inner wall defining the lumen, an outer wall coupled to the inner wall at the proximal end and the distal end, a cavity formed from the coupled inner wall and outer wall and configured to hold a deliverable substance, and a plurality of openings connected to the cavity and positioned on a distal-facing surface of the distal end. At least a portion of the inner wall or the outer wall may be a flexible wall that transitions between a first configuration and a second configuration to cause delivery of the deliverable substance from the cavity to a treatment site through the plurality of openings when a force is applied to the flexible wall.

In any of the exemplary delivery devices disclosed, the tube may further include a plurality of protrusions extending distally from the distal-facing surface. The plurality of protrusions may be positioned along one or both of an outer circumference of the distal-facing surface and an inner circumference of the distal-facing surface. The tube may further include a plurality of channels connecting the plurality of openings to the cavity. Each of the plurality of channels may include a proximal end connected to the cavity and a distal end connected to a respective one of the plurality of openings, and each of the plurality of channels may be a tapered channel that gradually narrows from the proximal end toward the distal end. Each of the plurality of openings may be configured to form a seal, and the seal may break when the force is applied to the flexible wall. A configuration of the lumen may prevent obstruction of components at the distal tip of the medical device when the tube is mounted to the medical device. The components at the distal tip of the medical device may include at least one of distal openings of one or more working channels, one or more illumination devices, or one or more visualization devices.

In some aspects, at least the portion of the inner wall may be the flexible wall, and the force applied to the flexible wall to cause the delivery of the deliverable substance may be an extension of the distal tip of the medical device through the lumen from the proximal end to the distal end. At least the portion of the inner wall may transition between a concave configuration and a straight configuration, and in the concave configuration, an innermost diameter of at least the portion of the inner wall may be less than an outer diameter of the distal tip of the medical device, causing at least the portion of the inner wall to transition from the concave configuration to the straight configuration when the distal tip of the medical device is extended through the lumen.

In other aspects, at least the portion of the outer wall may be the flexible wall, and the delivery device may further include a compression device surrounding a portion of the tube and configured to move from a first position to a second position along the tube. The force applied to the flexible wall to cause the delivery of the deliverable substance may be a movement of the compression device from the first position to the second position along the tube. At least the portion of the outer wall may transition between a convex configuration and a straight configuration, and in the convex configuration, an outermost diameter of at least the portion of the outer wall may be greater than an inner diameter of the compression device, causing at least the portion of the outer wall to transition from the convex configuration to the straight configuration as the compression device is moved from the first position to the second position.

In these other aspects, the delivery device may further include an actuation device, and one or more actuators connected to the compression device and extended to the actuation device. Actuation of the one or more actuators via the actuation device may move the compression device from the first position to the second position. In some examples, the one or more actuators may extend distally from the compression device, loop around the distal end of the tube into the lumen, and proceed proximally through a working channel of the medical device to extend to the actuation device. In other examples, the delivery device may further include a flange attached to the proximal end of the tube and having one or more through holes. The one or more actuators may extend from the compression device via the one or more through holes to the actuation device. The delivery device may further include one or more outer tubes coupled to the flange and enclosing the one or more actuators from a proximal surface of the flange to the actuation device. The tube may be formed from an assembly comprised of at least a first assembly component and a second assembly component. The first assembly component may include the inner wall and a proximal-facing surface of the proximal end. The second assembly component may include the outer wall and the distal-facing surface of the distal end.

In other examples, a delivery device may include a lumen extending from a proximal end to a distal end of the delivery device. The proximal end may be configured to be mounted to a distal tip of a medical device via the lumen. The delivery device may also include an inner wall defining the lumen. At least a portion of the inner wall may be a flexible wall having a concave configuration in a first position. The delivery device may further include an outer wall coupled to the inner wall at the proximal end and the distal end, a cavity formed from the coupled inner wall and outer wall and configured to hold a deliverable substance, and a plurality of openings connected to the cavity and positioned on a distal-facing surface of the distal end. When the delivery device is mounted to the distal tip of the medical device and the distal tip of the medical device is extended distally from the proximal end to the distal end through the lumen, at least the portion of the inner wall may transition from the first position to a second position having a substantially straight configuration. The transition may compress the cavity and cause delivery of the deliverable substance from the cavity to a treatment site through the plurality of openings.

In any of the exemplary delivery devices disclosed herein, the delivery device may further include a plurality of protrusions distally extending from the distal-facing surface. The plurality of protrusions may be positioned along at least one of an outer circumference or an inner circumference of the distal-facing surface.

In further examples, a delivery device may include an assembly forming a tube having a proximal end and a distal end that may be mountable to a medical device. The assembly may include a first assembly component including an inner wall of the tube and a proximal-facing surface of the proximal end, and a second assembly component coupled to the first assembly component at the proximal end and the distal end. The second assembly may include an outer wall of the tube and a distal-facing surface of the distal end. At least a portion of the outer wall may be a flexible wall transitionable between a convex configuration and a substantially straight configuration. The distal-facing surface may include a plurality of openings. The assembly may also include a cavity formed from the coupled first assembly component and the second assembly component. The cavity may be connected to the plurality of openings and configured to hold a deliverable substance. The delivery device may further include a compression device surrounding the assembly and movable from a first position to a second position along a radial exterior of the assembly, and one or more actuators connected to the compression device and configured to move the compression device from the first position to the second position. When the one or more actuators are actuated, at least the portion of the outer wall may transition from the convex configuration to the substantially straight configuration as the compression device is moved from the first position to the second position. The transition may compress the cavity and cause delivery of the deliverable substance from the cavity to a treatment site through the plurality of openings.

In any of the exemplary delivery devices disclosed herein, the delivery device may further include an actuation device connected to a proximal end of the one or more actuators. In some examples, the one or more actuators may extend distally from the compression device, loop around the distal end of the tube into a lumen defined by the inner wall of the tube, and proceed proximally through a working channel of the medical device to extend to the actuation device. In other examples, the delivery device may further include a flange connected to the proximal end of the assembly and having one or more through holes, and an actuation device connected to a proximal end of the one or more actuators, where the one or more actuators may extend from the compression device via the one or more through holes to the actuation device.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIGS. 1A-1C depict various views of a medical delivery system having a first exemplary delivery device.

FIGS. 2A-2D depict various views of a second exemplary delivery device.

FIGS. 3A-3D depict various views of a third exemplary delivery device.

### DETAILED DESCRIPTION

Bleeding in the GI tract may be caused by peptic ulcers or other similar sores that develop on the lining of the stomach and/or the upper portion of the small intestine of the GI tract. The ulcers may develop due to stomach acid from bacteria or use of anti-inflammatory drugs, for example, that damage the lining. Internal bleeding is a common complication of these types of ulcers, particularly when the ulcer develops at a site of a blood vessel. If left untreated, the ulcer may swell or scar, which may create a blockage in the GI tract. Additionally or alternatively, the ulcer may perforate the stomach or small intestine and infect the abdomen, causing peritonitis. A bleeding ulcer in the GI tract may lead to anemia, bloody vomit, and/or bloody stools.

As briefly mentioned above, bleeding in the GI tract is often challenging to control as it can be difficult to access a site of the bleeding. Current treatment methods for stopping or reducing bleeding in the GI tract include injection therapy, thermal therapy, and mechanical therapy, which are time intensive, cost intensive, and/or highly localized treatments. One exemplary mechanical treatment method includes application of a hemostatic device, such as a patch or mesh to the site of the bleeding. For example, the patch or mesh may be deployed to the site of the bleeding using an endoscope, and a liquid or semi-liquid adhesive may be applied to secure the patch or the mesh to the site until at least clots are formed to stop the bleeding.

An example hemostatic device may be comprised of nano fibers fabricated from electrospinning. The hemostatic device may be comprised of biocompatible material, such as nylon urethane, nylon, or latex, capable of forming a flexible membrane. Additionally or alternatively, the hemostatic device may be comprised of a bioadhesive material, such as chitosan, modified chitosan, cellulose, polyhydroxyethylmethacrylate (pHEMA), polyvinyl alcohol (PVA), polyethylene glycol (PEG), or composites thereof. In some examples, the hemostatic device may be comprised of polypropylene, polyester, and expanded polytetrafluoroethylene (ePTFE). In further examples, the patch or mesh component of the hemostatic device may be coated with a hydrogel. The adhesive applied may include fibrin glue. In some examples, the adhesive applied may be a two-component adhesive, such as fibrinogen and thrombin.

Conventionally, the adhesive may be delivered through an injection needle that is delivered distally through a working channel of the endoscope to reach the site of the bleeding. However, the injection needle is limited to delivering the adhesive at a single point or location. Resultantly, the injection needle may have to be repeatedly repositioned and/or actuated to deliver the adhesive to multiple points or locations throughout the site to help ensure that the patch or the mesh is sufficiently secured to the site, which may increase an overall procedure time.

Therefore, aspects of this disclosure are directed to medical delivery systems, devices, and related methods of use for distributed delivery of a deliverable substance, such as an adhesive, to a treatment site. An exemplary medical delivery system may include a delivery device mountable or otherwise couplable to a distal tip of a medical device, where the delivery device is configured to deliver a deliverable substance in the distributed manner. Various configurations of the delivery device may be implemented.

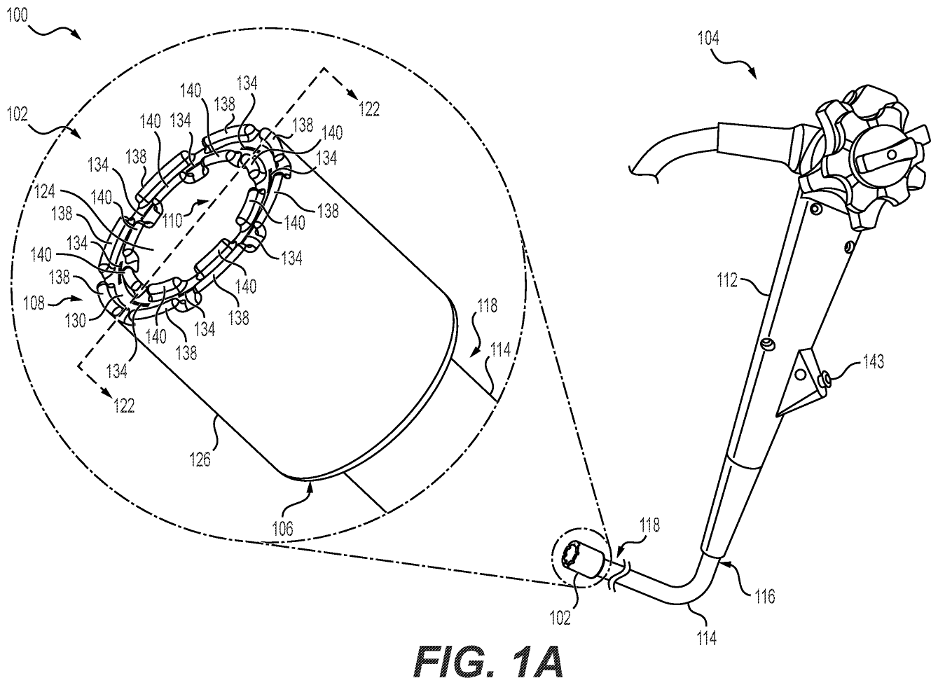

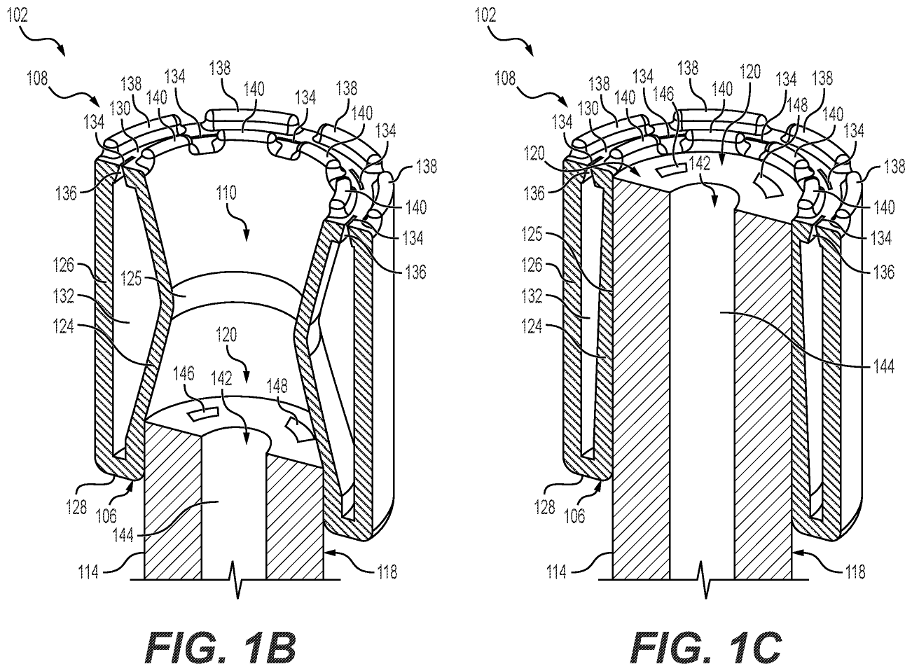

In one example and as described with reference to FIGS. 1A-1C below, a first delivery device may be a tube having a proximal end mountable to a distal tip of a medical device via a lumen that extends from the proximal end to a distal end of the tube. The tube may include an inner wall defining the lumen, and an outer wall coupled to the inner wall at the proximal end and the distal end. The coupled inner wall and outer wall form a cavity that is configured to hold the deliverable substance, and the tube may further include a plurality of openings connected to the cavity that are positioned on a distal-facing surface of the distal end of the tube. The inner wall may be a flexible wall having a substantially concave configuration in a first position and a substantially straight configuration in a second position. To deliver the deliverable substance, the distal tip of the medical device, to which the proximal end of the tube is mounted, may be extended distally from the proximal end to the distal end of the tube. As the distal tip of the medical device is extended distally, the flexible inner wall transitions from the first position to the second position. The transition compresses the cavity, which causes delivery of the deliverable substance from the cavity to a treatment site through the plurality of openings.

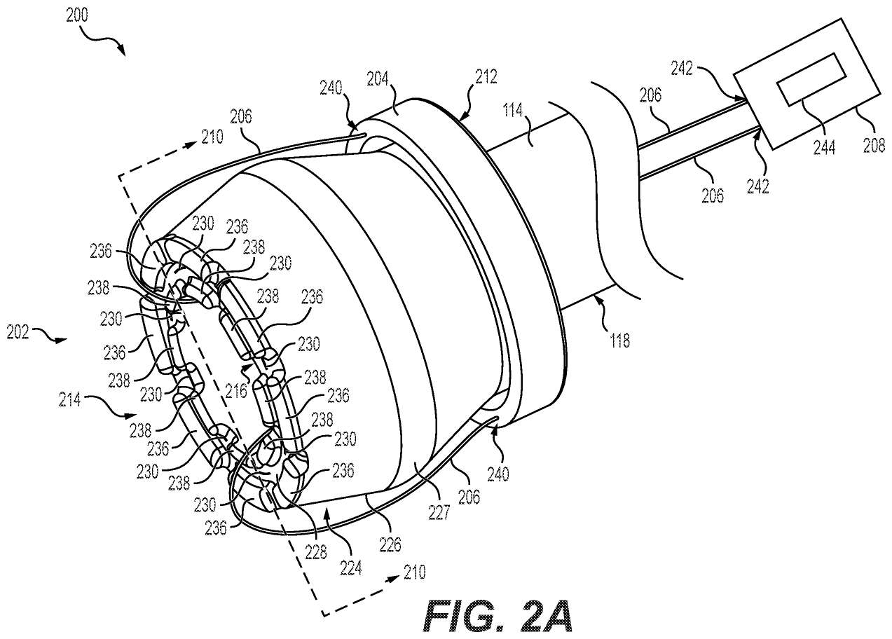

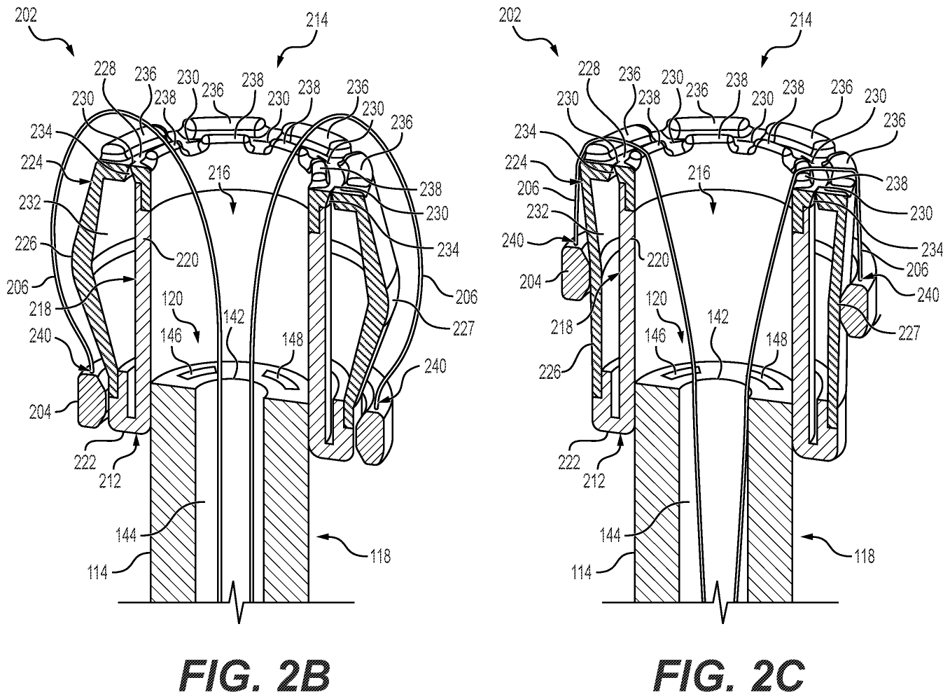

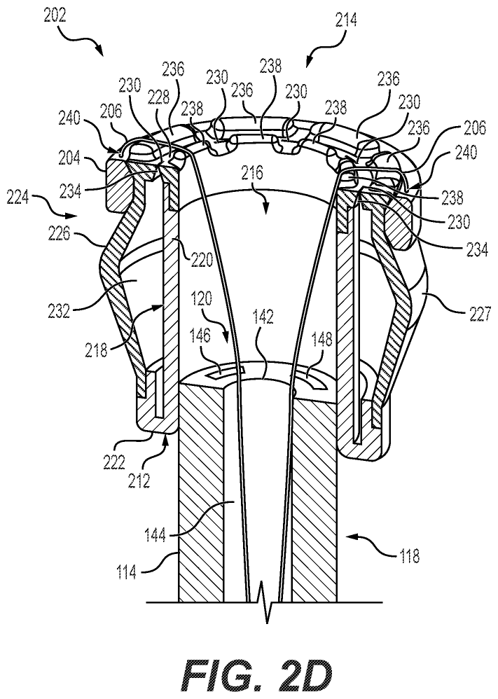

In another example and as described with reference to FIGS. 2A-2D below, a second delivery device may include an assembly forming a tube having a proximal end and a distal end, where the assembly is mountable to a distal tip of a medical device. The assembly includes a first assembly component and a second assembly component. The second assembly component may be coupled to the first assembly component at the proximal end and at the distal end of the assembly to form the tube and a cavity within the tube that is configured to hold a deliverable substance. The first assembly component may include an inner wall of the tube and a proximal-facing surface of the proximal end. The second assembly component may include an outer wall of the tube and a distal-facing surface of the distal end having a plurality of openings connected to the cavity. The outer wall may be a flexible wall that is configured to transition between a substantially convex configuration and a substantially straight configuration. The second delivery device may also include a movable compression device surrounding the tube, and one or more actuators connected to the compression device in a first configuration to move the compression device from a first position at the proximal end of the tube to a second position at the distal end of the tube. To deliver the deliverable substance, the one or more actuators are actuated, and the flexible outer wall transitions from the substantially convex configuration to the substantially straight configuration as the compression device is moved distally from the first position to the second position. The transition compresses the cavity, which causes delivery of the deliverable substance from the cavity to a treatment site through the plurality of openings.

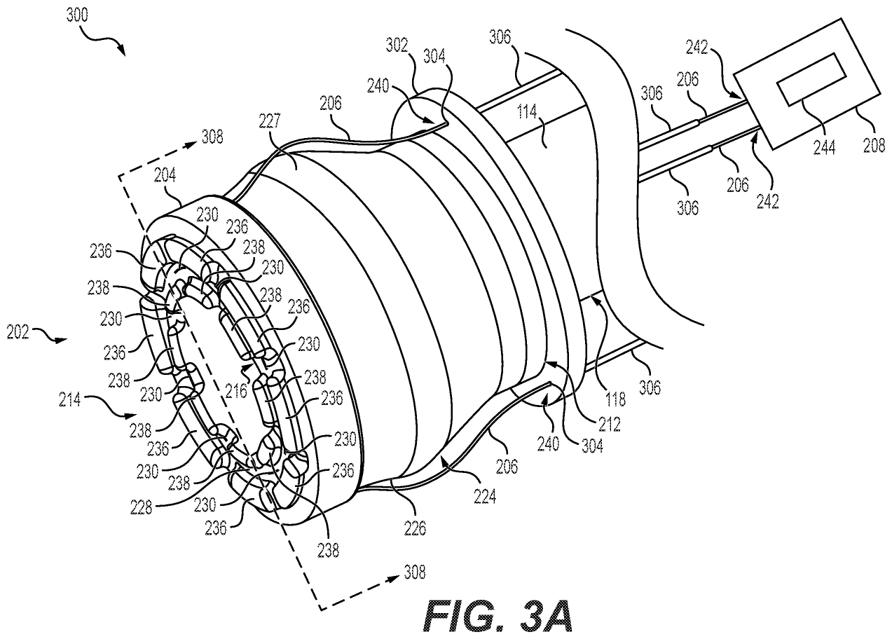

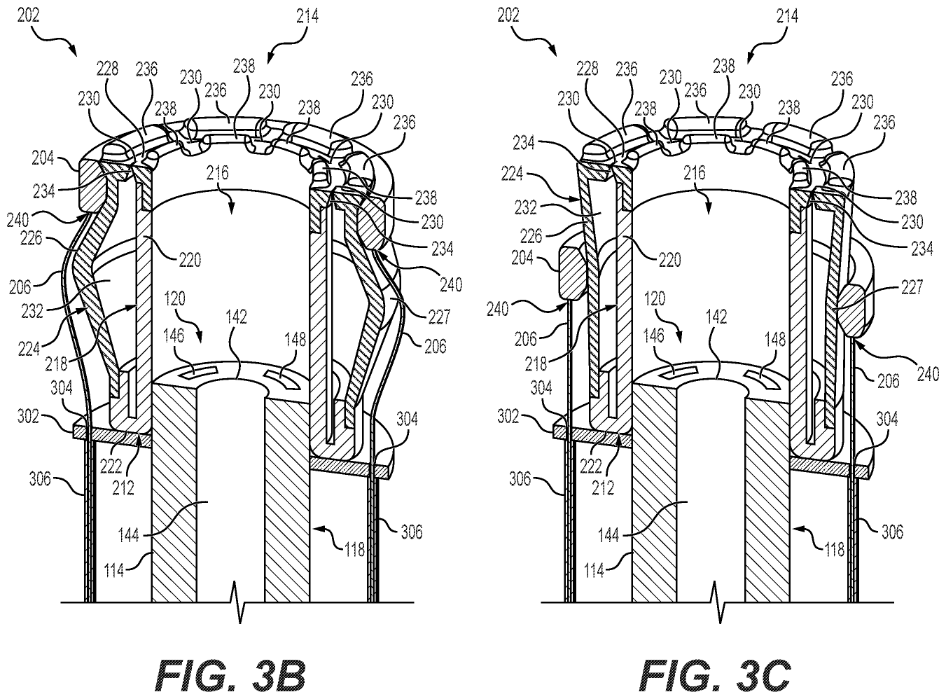

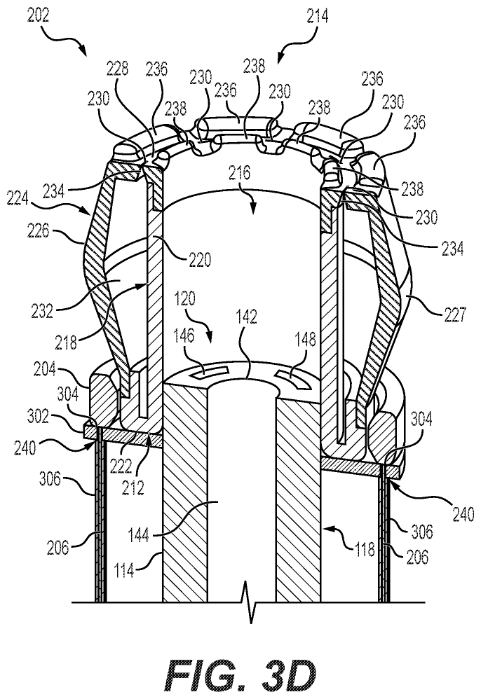

In a further example and as described with reference to FIGS. 3A-3D below, a third delivery device may be similar to the second delivery device, except the actuators are connected to the compression device in a different, second configuration. In the second configuration, the actuators move the compression device from a first position at the distal end of the tube to a second position at the proximal end of the tube. To deliver the deliverable substance, the one or more actuators are actuated, and the flexible outer wall transitions from the substantially convex configuration to the substantially straight configuration as the compression device is moved proximally from the first position to the second position. The transition compresses the cavity, which causes delivery of the deliverable substance from the cavity to a treatment site through the plurality of openings.

For any of the above-discussed exemplary delivery devices, the deliverable substance is distributable over a wider surface area at the treatment site with a single delivery. Additionally, when the delivery device or assembly of the delivery device is mounted or otherwise coupled to the distal tip of medical device, a working channel conventionally used to deliver an injection needle for adhesive delivery may be unobstructed. Therefore, the unobstructed working channel may be used to deliver other components concurrently and/or closer in time with the deliverable substance during a treatment procedure, which may increase efficiency and thus reduce an overall time of the procedure.

FIG. 1A depicts an exemplary medical delivery system 100. Medical delivery system 100 may include a delivery device 102 and a medical device 104. As shown, delivery device 102 may be removably mounted to medical device 104. Delivery device 102 may be a tube having a proximal end 106, a distal end 108, and a lumen 110 extending from proximal end 106 to distal end 108. Medical device 104 may include a handle 112 and a shaft 114 having a proximal end 116 coupled to handle 112 and a distal end 118 coupled to delivery device 102. For example, proximal end 106 of delivery device 102 may mount to or otherwise receive a distal tip 120 (See FIGS. 1B and 1C) at distal end 118 of medical device 104 via lumen 110. Medical device 104 may be moveable from proximal end 106 to distal end 108 of delivery device 102 through lumen 110. Movement of medical device 104 relative to delivery device 102 may cause delivery of a deliverable substance contained within delivery device 102, as described in detail below.

FIG. 1B depicts a cross-sectional view of delivery device 102 and distal tip 120 of shaft 114 taken along line 122 in FIG. 1A when distal tip 120 is positioned at proximal end 106 of delivery device 102. FIG. 1C depicts a cross-sectional view of delivery device 102 and distal tip 120 of shaft 114 taken along line 122 in FIG. 1A when distal tip 120 has been moved or extended through lumen 110 and is positioned at distal end 108 of delivery device 102.

Referring concurrently to FIGS. 1A-1C, delivery device 102 may be a hollow cylindrical component including an inner wall 124 and an outer wall 126. Although various components of delivery device 102 are discussed as being generally cylindrical in shape, this disclosure is not so limited. In other examples, components of delivery device 102 may be ovular, triangular, square, rectangular, or pentagonal, among other shapes.

Inner wall 124 may define lumen 110 through which distal tip 120 is received and extended. At least a portion of inner wall 124 is a flexible wall comprised of flexible material, such as silicon rubber, or any other flexible, biocompatible material. Based on this flexibility, inner wall 124 may transition between a first position having a first configuration and a second position having a second configuration in response to application of a force to inner wall 124. For example, when inner wall 124 is in the first position, the first configuration of inner wall 124 may be a substantially concave configuration defining an hourglass-shaped lumen 110, as shown in FIG. 1B. In the substantially concave configuration, a smallest inner diameter of inner wall 124 (e.g., a smallest diameter of lumen 110) is less than an outer diameter of distal tip 120. For example, portion 125 of inner wall 124 may have the smallest inner diameter inner wall 124. Accordingly, an extension of distal tip 120 distally through lumen 110 applies a force to inner wall 124 causing inner wall 124 to be pushed outward. As a result, inner wall 124 transitions from the first position having the substantially concave configuration to the second position having a substantially straight configuration, as shown in FIG. 1C.

Outer wall 126 may be a rigid wall comprised of plastic or any other rigid, biocompatible material. Outer wall 126 and inner wall 124 may be coupled to one another at proximal end 106 and distal end 108 of delivery device 102 to form a proximal-facing surface 128 of the tube at proximal end 106 and a distal-facing surface 130 of the tube at distal end 108. In some examples, proximal-facing surface 128 and/or distal-facing surface 130 may include a first surface portion formed from inner wall 124 and a second surface portion coupled to the first surface portion that is formed from outer wall 126.

A cavity 132 (FIGS. 1B and 1C) may be formed from the coupled inner wall 124 and outer wall 126. For example, cavity 132 may be defined by inner wall 124, outer wall 126, proximal-facing surface 128, and distal-facing surface 130. Cavity 132 may be configured to contain a deliverable substance that is delivered via delivery device 102 to a treatment site. For example, the deliverable substance may be an adhesive that is delivered and applied to a hemostatic device (e.g., a patch or a mesh) deployed or otherwise positioned at a treatment site. Example deliverable substances include, but are not limited to, thrombin sealant, fibrin glue, bovine serum albumin-glutaraldehyde, and/or gelatin matrices. In some examples, within an interior of cavity 132, delivery device 102 may include two or more divider walls extending from inner wall 124 to outer wall 126 and from proximal-facing surface 128 to distal-facing surface 130 to form two or more compartments within cavity 132. Each compartment may be configured to contain a different type of deliverable substance. For example, the deliverable substance may be comprised of a first component and a second component that may be mixed at the treatment site. In some examples, when the deliverable substance is an adhesive, the first and second components may include a resin and a hardener that mix at the treatment site, for example, to form a polymeric adhesive, which may have high adhesion and/or holding strength. A first compartment and a second compartment formed by the two divider walls may respectively contain the first component and the second component.

Distal-facing surface 130 may include a plurality of openings 134 (e.g., holes, slots, slits, or the like) spaced apart along a circumference of distal-facing surface 130 that are connected to cavity 132. The deliverable substance may be delivered from cavity 132 to the treatment site via each of openings 134 when the deliverable substance is pressurized due to compression of cavity 132. For example, as distal tip 120 is moved or extended distally through lumen 110, and inner wall 124 transitions from the first position (FIG. 1B) having the substantially concave configuration to the second position (FIG. 1C) having the substantially straight configuration, cavity 132 may be compressed (e.g., the volume of space within cavity 132 decreases). As cavity 132 compresses, the deliverable substance contained therein becomes increasingly pressurized, and flows distally from cavity 132 via openings 134. Delivery of the deliverable substance from each of openings 134 enables the deliverable substance to be distributed over or contemporaneously delivered to a larger surface area of the treatment site (or of a device deployed at the treatment site) as compared to conventional injection-based delivery that is limited to application at a single point or location.

In some examples, openings 134 may be connected to cavity 132 via a plurality of channels 136 corresponding to openings 134 (e.g., openings 134 and channels 136 may form nozzles) to control flow of the deliverable substance. For example, each channel 136 may include a proximal channel end connected to cavity 132 and a distal channel end connected to a respective opening 134. As shown, channels 136 may be tapered channels that gradually narrow from the proximal channel end toward the distal channel end. One side of channels 136 extending from proximal channel end to distal channel end may be formed or defined by rigid material of outer wall 126. The other, opposing side of channels 136 extending from proximal channel end to distal channel end may be formed or defined by flexible material of inner wall 124. For example, the outermost side relative to the tube maybe rigid, while the innermost side relative to the tube may be flexible.

Additionally, openings 134 may be configured to form a seal such that openings 134 are completely closed when inner wall 124 is in the first position having the substantially concave configuration. As distal tip 120 is extended distally through lumen 110 and inner wall 124 transitions to the second position, the increasingly pressurized deliverable substance, may break or open the seal, causing openings 134 to open to allow the deliverable substance to flow distally from cavity 132 via openings 134. In some examples, at least a portion of each of openings 134 are formed from the flexible material of inner wall 124 to facilitate the formation of the seal. For example, at least a portion of openings 134 are disposed on the first surface portion of distal-facing surface 130 formed from inner wall 124. The seal individually and/or in combination with the configuration of openings 134 and channels 136 may help to prevent undesired leakage of the deliverable substance from cavity 132. For example, the seal may help to prevent inadvertent delivery of the deliverable substance, and thus may also help to prevent inadvertent contact of the deliverable substance with bodily fluids during delivery or positioning of medical device 104 at the treatment site.

A plurality of protrusions, including a first set of protrusions 138 and a second set of protrusions 140, may extend distally from distal-facing surface 130 of delivery device 102. Prior to delivery of the deliverable substance, delivery device 102 may be positioned such that first and second set of protrusions 138, 140 contact the treatment site and/or a device deployed or otherwise positioned at the treatment site, such as a hemostatic device described in detail below. First and second set of protrusions 138, 140 may create a spacing (e.g., a vertical space) between the treatment site and/or hemostatic device and openings 134. This spacing may help to facilitate the distribution of the adhesive over the larger surface area of the treatment site and/or the hemostatic device.

First set of protrusions 138 may be spaced along an outer or outermost circumference of distal-facing surface 130. For example, first set of protrusions 138 may extend distally from the second surface portion of distal-facing surface 130 comprised of rigid, outer wall 126. In some examples, first set of protrusions 138 may be comprised of a same or similar material as outer wall 126. Second set of protrusions 138 may be spaced along an inner or innermost circumference of distal-facing surface 130. For example, second set of protrusions 140 may extend distally from the first surface portion of distal-facing surface 130 comprised of flexible, inner wall 124. In some examples, second set of protrusions 140 may be comprised of a same or similar material as inner wall 124.

As shown, first set of protrusions 138 may correspond to second set of protrusions 140. For example, each protrusion in first set of protrusions 138 may circumferentially align with a protrusion in second set of protrusions 140. The corresponding protrusions in first and second set of protrusions 138, 140 may be positioned between openings 134 along distal-facing surface 130. However, in other examples, first set of protrusions 138 may be staggered or otherwise offset from second set of protrusions 140. In some examples, first and second set of protrusions 138, 140 may have protrusions of equal height, length, and width dimensions. In other examples, protrusions in first set of protrusions 138 may have different height, length, and/or width dimensions than protrusions in second set of protrusions 140.

Optionally, a gasket may be positioned at distal end 108 of delivery device 102. The optional gasket may be generally shaped as a substantially circular or cylindrical cap positioned over distal end 108. A proximal end of the optional gasket may conform to inner wall 124. Optional gasket may then extend distally to distal-facing surface 130. A distal end of the gasket positioned on distal-facing surface 130 may include a plurality of flaps. Each of the flaps may be positioned between first set and second set of protrusions 138, 140 extending distally from distal-facing surface 130. The flaps may be configured to provide further cover, closure, and/or sealing of openings 134 positioned between first and second set of protrusions 138,140. In these aspects, the flaps may help to prevent leakage or inadvertent flow of the deliverable substance from openings 134 when inner wall 124 is in the first position having the substantially concave configuration (e.g., prior to movement of distal tip 120 from proximal end 106 to distal end 108 through lumen 110). As distal tip 120 is extended distally through lumen 110, the increasingly pressurized deliverable substance, as described in detail above, may force the flaps to open distally to allow the deliverable substance to flow distally from cavity 132 via openings 134. The above-described spacing (e.g., a vertical space) from the hemostatic device created by the first and second set of protrusions 138,140 may enable the flaps to open distally within the spacing to help facilitate the flow and/or distribution of the deliverable substance.

The deliverable substance contained in cavity 132 may be a liquid or a semi-liquid substance that is capable of flowing out of delivery device 102. When the deliverable substance is an adhesive for delivery to a hemostatic device deployed or otherwise positioned at the treatment site, the adhesive may be any type of adhesive that is absorbable by the hemostatic device and has a property of adhesion between the hemostatic device and tissue at the treatment site. In these aspects, the adhesive may bind the hemostatic device to the tissue. Cavity 132 may be pre-filled with the deliverable substance during a manufacturing of delivery device 102. Additionally or alternatively, cavity 132 may be filled by the operator of the medical delivery system 100 (or the operator's assistant) prior to delivery device 102 being mounted onto medical device 104 during the treatment procedure. For example, a syringe or other similar instrument may be used to transfer the deliverable substance from a separate storage container to cavity 132 via one of openings 134.

Medical device 104 may be an endoscope or other type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, or catheter. The configuration of delivery device 102 may help to prevent obstruction of components at distal tip 120 of medical device 104 when delivery device 102 is mounted to medical device 104. Resultantly, the components at distal tip 120 may operate per usual. Exemplary components at distal tip 120 may include distal openings 142 of one or more working channels 144 and/or an imaging system, including one or more illumination devices 146 and/or one or more visualization devices 148. Working channels 144 may be configured to supply fluid, apply suction, and/or deliver instruments to the treatment site via the distal openings 142. Medical device 104 may include one or more ports 143 located on handle 112 that provide a connection of a fluid or aspiration source to working channels 144 and/or an entry for instruments to be inserted into working channels 144. Illumination devices 146 (e.g., one or more LEDs, optical fibers, and/or other illuminators) of the imaging system may be configured to illuminate areas of the patient's body during the procedure to, e.g., facilitate imaging. Visualization devices 148 (e.g., one or more cameras, one or more image sensors, endoscopic viewing elements, optical assemblies including one or more image sensors and one or more lenses, etc.) of the imaging system may be configured to capture images during the treatment procedure.

To provide a non-limiting, illustrative method of use of medical delivery system 100, during an exemplary treatment procedure, delivery device 102 may be mounted to medical device 104. Medical device 104 may be inserted into a body lumen of the patient and navigated toward a treatment site. In one example, the treatment site may be a bleeding site in the stomach or duodenum, and medical device 104 may be inserted into the patient's mouth and navigated through the esophagus into the stomach and/or through the stomach and into the duodenum to the treatment site. The imaging system of medical device 104 (e.g., illumination devices 146 and/or visualization devices 148) may provide visual guidance as medical device 104 is navigated to the treatment site.

In one example, a hemostatic device (e.g., a patch or a mesh) may have been previously delivered to the treatment site, and deployed to cover the treatment site. In some examples, more than one hemostatic device may have been delivered and deployed (e.g., based on a size of the treatment site). For example, prior to mounting delivery device 102 to medical device 104, medical device 104 (or another medical device similar to medical device 104) may have been inserted into the body lumen of the patient to deliver the hemostatic device, e.g., via working channel 144. In some examples, a hemostatic device delivery system may be used in conjunction with medical device 104 to deploy the hemostatic device at the treatment site. For example, the hemostatic device delivery system may be delivered through and extend distally from distal opening 142 of working channel 144 that is positioned at the treatment site to deploy the hemostatic device. In examples, where more than one hemostatic device is needed, the hemostatic device delivery system may be configured to deploy each of the hemostatic devices sequentially and/or concurrently. Once the hemostatic device was deployed, medical device 104 may have been removed from the patient to enable delivery device 102 to be mounted to medical device 104. Alternatively, if the other medical device similar to medical device 104 was used for hemostatic device delivery and deployment, the other medical device may be removed from patient and replaced with medical device 104 having delivery device 102 mounted thereto.

In another example, medical device 104 having delivery device 102 mounted thereto, may be configured to facilitate delivery and deployment of the hemostatic device, e.g., via working channel 144, just prior to the delivery of the deliverable substance (e.g., an adhesive) via delivery device 102. In such examples, only one insertion (and removal) of medical device 104 from the body lumen of the patient may be necessary to deliver both the hemostatic device and deliverable substance, which may help to decrease an overall procedure time. Additionally, delivery device 102 may include one or more markings or other visual features to help guide the operator to mount delivery device 102 to medical device 104. The one or more markings or other visual features on delivery device 102 may help the operator to position delivery device 102 in a manner that helps to ensure distal opening 142 of working channel 144 remains unobstructed. In a further example, an end cap and/or a tube through which the hemostatic device may be delivered may be positioned around (e.g., around a radial exterior of delivery device 102) or adjacent to delivery device 102 and/or medical device 104.

For delivery of the deliverable substance, a distalmost end of first and second set of protrusions 138, 140 of delivery device 102 may be placed in contact with or adjacent to the hemostatic device. Then, the operator may manipulate medical device 104 to cause distal tip 120 to be moved or extended from proximal end 106 to distal end of delivery device 102 via lumen 110. As distal tip 120 is moved or extended distally, flexible inner wall 124 transitions from the first position having the substantially concave configuration, as shown in FIG. 1B, to the second position having the substantially straight configuration, as shown in FIG. 1C. The transition of inner wall 124 to the second position compresses cavity 132 (e.g., the volume of space within cavity 132 decreases), causing the deliverable substance contained therein to become increasingly pressurized. The pressurized deliverable substance breaks the seal of (e.g., opens previously closed) openings 134, and the deliverable substance flows distally from cavity 132 via openings 134 onto the hemostatic device. The deliverable substance may be delivered in a distributed manner over the area of the treatment site to help secure the hemostatic device to the treatment site to, e.g., control bleeding. The distributed delivery may be further enhanced by the vertical space between openings 134 and hemostatic device created by protrusions 138, 140.

While the specific application of medical delivery system 100 described herein is associated with a treatment procedure in which an adhesive is distributed to a hemostatic device deployed at a bleeding site within the GI tract, medical delivery system 100 is not limited to this application. For example, medical delivery system 100 may be used for any procedure in which a liquid or semi-liquid substance is to be delivered in a distributed manner to a site within a patient's body this is accessible via a medical device, such as medical device 104.

FIGS. 2A-2D depict various views of a second exemplary delivery device 200. For example, instead of delivery device 102, medical delivery system 100 shown in FIG. 1A may include delivery device 200. Delivery device 200 may include an assembly 202, a compression device 204 movable along assembly 202, one or more actuators 206 for moving compression device 204, and an actuation device 208 to actuate actuators 206. Assembly 202, similar to delivery device 102, may be removably mounted to distal end 118 of shaft 114 of medical device 104 (FIG. 1A). In some examples, actuation device 208 may be connected to (e.g., mounted on) handle 112 (FIG. 1A), as described in further detail below.

FIG. 2A depicts a side perspective view of delivery device 200 when assembly 202 is mounted to distal end 118 of shaft 114. FIGS. 2B-2D depict cross-sectional views of a distal portion of delivery device 200 and distal tip 120 of shaft 114 taken along line 210 in FIG. 2A when compression device 204 is in a respective first position, second position, and third position relative to assembly 202. Referring concurrently to FIGS. 2A-2D, assembly 202 may be a two-component assembly that forms a tube having a proximal end 212, a distal end 214, and a lumen 216 extending from proximal end 212 to distal end 214. Proximal end 212 of assembly 202 may mount to or otherwise receive distal tip 120 at distal end 118 of shaft 114 via lumen 216.

A first assembly component 218 of assembly 202 may include an inner wall 220 and a proximal-facing surface 222, with proximal-facing surface being positioned at proximal end 212. First assembly component 218 may be a rigid component comprised of, for example, plastic or any other rigid, biocompatible material. Inner wall 220 may define lumen 216 that receives distal tip 120, and distal tip 120 may be movable or extendable through lumen 110. The configuration of inner wall 220 of assembly 202 may help to prevent obstruction of components at distal tip 120 of medical device 104, including distal opening(s) 142 of one or more working channels 144, illumination device(s) 146, and/or visualization device(s) 148, when assembly 202 is mounted to medical device 104, and thus the components may operate per usual, as describe in detail above with respect to FIGS. 1A-1C.

A second assembly component 224 of assembly 202 may include an outer wall 226 and a distal-facing surface 228, with distal-facing surface 228 being positioned at distal end 214. Second assembly component 224 may be a flexible component comprised of, for example, silicon rubber or any other flexible, biocompatible material. In a first position (e.g., when no force is being applied to outer wall 226), outer wall 226 may have a substantially convex configuration as shown in FIGS. 2A, 2B, and 2D. Based on the flexibility of outer wall 226, outer wall 226 may be transitionable between the first position and a second position having a substantially straight configuration in response to application of a force to the flexible wall (e.g., outer wall 226), as shown in FIG. 2C.

Distal-facing surface 228 may include a plurality of openings 230 that are spaced apart along the circumference of distal-facing surface 228. Openings 230 may be similar to openings 134 shown in and described with reference to FIGS. 1A-C, except that an entirety of openings 134 may be disposed on flexible material (e.g., the flexible material from which second assembly component 224 is comprised). Distal-facing surface 228 may also include a first set of protrusions 236 that are spaced apart along the outermost circumference of distal-facing surface 130. First set of protrusions 236 may be similar to first set of protrusions 138 shown in and described with reference to FIGS. 1A-1C, except that first set of protrusions 236 may be comprised of flexible material (e.g., the flexible material from which second assembly component 224 is comprised). Distal-facing surface 228 may further include a second set of protrusions 238 extending distally from distal-facing surface 228. Similar to second set of protrusions 140 shown in and described with reference to FIGS. 1A-1C, second set of protrusions 238 may be comprised of flexible material and spaced apart along the innermost circumference of distal-facing surface 228. Having first and second sets of protrusions 236, 238 that are comprised of flexible material may help to reduce trauma to tissue at the treatment site when the delivery device 200 is positioned in contact with the tissue (e.g., via the first and second sets of protrusions 236, 238). Additionally, the flexible first and second sets of protrusions 236, 238 may reduce an amount of friction generated by actuators 206 when actuated to move compression device 204.

First assembly component 218 and second assembly component 224 may be separately manufactured and coupled to one another at proximal end 212 and distal end 214. First assembly component 218 and second assembly component 224 may be mechanically coupled, adhered (e.g., via glue or other adhesive), ultrasonically welded, and/or otherwise affixed to one another. A cavity 232 formed from the coupled first assembly component 218 and the second assembly component 224 may be configured to hold a deliverable substance, such as the same or similar types of deliverable substances configured to be held by cavity 132 described above with reference to FIGS. 1A-1C. Cavity 232 may be connected to openings 230 to enable the deliverable substance to be delivered via openings 230 when cavity 232 is compressed. In some examples, openings 230 may be connected to cavity 232 via a plurality of channels 234 corresponding to openings 230. Channels 234 may be similar to channels 136 shown in and described with reference to FIGS. 1A-1C, for example, except that both sides of channels 234 may be formed or defined by flexible material (e.g. the flexible material from which second assembly component 224 is comprised). Formation of both sides of channels 234 from flexible first and second sets of protrusions 236, 238 may help enable flow of the deliverable substance in a distal direction that is perpendicular to the distal-facing surface 228.

Compression device 204 may be an annular ring comprised of a rigid material, such as, for example, plastic or any other rigid, biocompatible material. Accordingly, an inner diameter of compression device 204 may be fixed. In some examples, compression device 204 may be comprised of a same material as first assembly component 218. Compression device 204 may be configured to initially surround assembly 202 at proximal end 212, and may be moved distally from proximal end 212 to distal end 214 of assembly 202 (e.g., by actuators 206) to cause delivery of the deliverable substance.

Actuators 206 may include wires. Additionally or alternatively, actuators 206 may include cables, coils, threads, or other similar structures. Actuators 206 may be connected to compression device 204, and are configured to move (e.g., pull) compression device 204 from proximal end 212 to distal end 214 of assembly 202 upon actuation by actuation device 208. As shown, at least two actuators 206 may be connected to opposing first and second sides of compression device 204 to facilitate smooth, even movement of compression device 204. In other examples, three or more actuators 206 (e.g., four actuators 206) may be connected to and spaced (e.g., evenly or unevenly spaced) along compression device 204.

As shown, a first end 240 of each of actuators 206 may be connected to a distal surface of compression device 204. However, in other examples, first end 240 of actuators 206 may be coupled to other surfaces of compression device 204 (e.g., interior, exterior, or proximal surfaces). Mechanical connections, adhesive-based mechanisms, and/or injection molding, among other examples, may be used to connect first end 240 of actuators 206 to compression device 204. A second end 242 of each of actuators 206 may be connected to actuation device 208.

Actuators 206 may extend from compression device 204 to actuation device 208 through working channel 144 of medical device 104 (e.g., a first configuration of actuators 206 relative to compression device 208 and actuation device 208). For example, actuators 206 may initially extend distally from the connection point to compression device 204 to distal end 214 of assembly 202, loop around assembly 202, proceed proximally through lumen 216 into distal opening 142 of working channel 144, extend proximally through working channel 144 into handle 112 (FIG. 1A), exit handle 112 through port 143 (FIG. 1A), and connect to actuation device 208. For example, second end 242 of actuators 206 may include a knot, loop, or other similar structure that may be received by a hook of actuation device 208 to secure actuators 206 to actuation device 208. Actuation device 208 may also include an actuation mechanism 244. Actuation mechanism 244 may be actuated by an operator to cause actuators 206 to be pulled proximally to move compression device 204 distally from proximal end 212 to distal end 214 of assembly 202. An exemplary actuation mechanism 244 may include a rotating mechanism (e.g., a roller device), a lever mechanism, a sliding mechanism, a pushing mechanism, a pulling mechanism, or the like. In some examples, second end 242 of actuators 206 may be coupled to actuation mechanism 244 within actuation device 208 such that movement or operation of actuation mechanism 244 controls actuation of actuators 206.

Actuation device 208 may be removably mounted to or disposed on handle 112 of medical device 104. Mounting actuation device 208 on handle 112 of medical device 104 may enable an operator of medical device 104 to more easily operate both medical device 104 and delivery device 200 simultaneously, for example, optionally using a single hand of the operator that is holding handle 112. In other examples, actuation device 208 may be separately held from medical device 104. For example, actuation device 208 may include a handle for gripping by the operator (e.g., the operator that is holding handle 112 or another operator).

FIGS. 2B-2D show three exemplary positions of compression device 204 prior to and as compression device 204 is moved from proximal end 212 to distal end 214 of assembly 202 to cause delivery of the deliverable substance. In a first position prior to actuation of actuators 206 shown in FIG. 2B (also shown in FIG. 2A), compression device 204 may be configured to surround assembly 202 at proximal end 212. For example, the fixed inner diameter of compression device 204 may be greater than an outer diameter of assembly 202 at proximal end 212. In some examples, compression device 204 may be sized to fit securely around assembly 202 at proximal end 212 such that compression device 204 maintains the first position until actuators 206 are actuated. Additionally or alternatively, although not shown, a fastening or securement mechanism, such as an outwardly protruding wall at proximal end of assembly 202 and a corresponding notch on interior surface of compression device 204, may secure compression device 204 in the first position until actuators 206 are actuated. When compression device 204 is in the first position, outer wall 226 may be in the first position having the substantially convex configuration.

After a hemostatic device has been deployed or otherwise positioned at a treatment site as described in detail above with reference to FIGS. 1A-1C, a distalmost end of first and second set of protrusions 236, 238 of assembly 202 may be placed in contact with or adjacent to the hemostatic device. Then, the operator may actuate (e.g., by rotating, sliding, pushing, and/or pulling) actuation mechanism 244 on actuation device 208 to cause delivery of the deliverable substance. For example, actuation of actuation mechanism 244 may pull or urge actuators 206 proximally, causing compression device 204 to move distally from the first position to a second position, as shown in FIG. 2C.

In the second position, compression device 204 is positioned between proximal end 212 and distal end 214, and surrounds a portion 227 of outer wall 226 of assembly 202 having an outer diameter in the substantially convex configuration greater than the fixed inner diameter of compression device 204. Due to the difference in diameters and flexibility of outer wall 226, as compression device 204 is moved toward the second positon, compression device 204 applies force to outer wall 226, causing outer wall 226 to be pushed inward. As a result, outer wall 226 transitions from the first position having the substantially convex configuration, as shown in FIG. 2B, to the second position having the substantially straight configuration, as shown in FIG. 2C. The transition of outer wall 226 to the substantially straight configuration compresses cavity 232 (e.g., the volume of space within cavity 232 decreases), causing the deliverable substance contained therein to become increasingly pressurized, and to flow distally from cavity 232 via openings 230.

Delivery of the deliverable substance from each of openings 230 enables the deliverable substance to be distributed over or contemporaneously delivered to a larger surface area of the hemostatic device or treatment site, as compared to a conventional injection-based delivery that is limited to application at a single point or location. The distributed delivery may be further enhanced by the vertical space between openings 230 and hemostatic device, for example, created by first and second set of protrusions 236, 238.

As the deliverable substance is being delivered, actuators 206 may continue to be pulled proximally causing compression device 204 to move from the second position to a third position, as shown in FIG. 2D. In the third position, compression device 204 is located at distal end 214 of assembly 202, and surrounds a portion of outer wall 226 of assembly 202 having an outer diameter less than the fixed inner diameter of compression device 204 (e.g., similar to the first position). In some examples, compression device 204 may be sized to fit securely around assembly 202 at distal end 214 (similar to at proximal end 212), such that compression device 204 maintains the third position and cannot be moved any further distally to prevent compression device 204 from disconnecting from assembly 202. Additionally or alternatively, although not shown, a fastening or securement mechanism, such as an outwardly protruding wall at distal end 214 of assembly 202 and a corresponding notch on interior surface of compression device 204, may help to secure compression device 204 in the third position.

As compression device 204 moves from the second position to the third position, and passes the portion 227 of outer wall 226 having the outer diameter in the substantially convex configuration greater than the fixed inner diameter of compression device 204, the force applied by compression device 204 is at least partially released. As a result, outer wall 226 may at least partially transition back from the second position having the substantially straight configuration, shown in FIG. 2C, to the first position having the substantially convex configuration, shown in FIG. 2D. In some examples, outer wall 226 may have resilient and/or shape memory properties, which may further help to retain compression device 204 in the third position.

FIGS. 3A-3D depict various views of a third exemplary delivery device 300. For example, instead of delivery device 102, medical delivery system 100 shown in FIG. 1A may include delivery device 300. Delivery device 300 may be similar to delivery device 200. For example, delivery device 300 may include assembly 202 mountable to distal end 118 of shaft 114 of medical device 104, compression device 204 movable along assembly 202, actuators 206 for moving compression device 204, and actuation device 208 for actuating actuators 206 via actuation mechanism 244. As described in detail above with reference to FIGS. 2A-2D, assembly 202 includes rigid, first assembly component 218 comprised of inner wall 220 and proximal-facing surface 222, and flexible, second assembly component 224 comprised of outer wall 226 and distal-facing surface 228, which includes openings 230. Additionally, assembly 202 includes cavity 232 formed from the coupling of first assembly component 218 and second assembly component 224, channels 234 connecting cavity 232 to openings 230, and first and second set of protrusions 236, 238 extending distally from distal-facing surface 228.

However, different from delivery device 200, delivery device 300 may also include a flange 302 having one or more through holes 304, and one or more outer tubes 306. Additionally, actuators 206 extend from compression device 204 to actuation device 208 in a different, second configuration causing actuators 206 to move compression device 204 in a different direction along assembly 202 (e.g., from distal end 214 to proximal end 212).

FIG. 3A depicts a side perspective view of delivery device 300 mounted to distal end 118 of shaft 114 of medical device 104. FIGS. 3B-3D depict cross-sectional views of a distal portion of delivery device 300 and distal tip 120 of shaft 114 taken along line 308 in FIG. 3A when compression device 204 is in a respective first position, second position, and third position relative to assembly 202. Referring concurrently to FIGS. 3A-3D, flange 302 may be attached to proximal end 212 of assembly 202, and more specifically to proximal-facing surface 222 of first assembly component 218. In other examples, flange 302 may be manufactured as part of first assembly component 218 (e.g., proximal-facing surface of flange 302 is a proximalmost-facing surface of first assembly component 218). Flange 302 may be a hollow circular or cylindrical component comprised of a rigid material, such as, for example, plastic or any other biocompatible, rigid material. Flange 302 may extend radially outward beyond proximal-facing surface 222.

Flange 302 may include through holes 304 for receiving actuators 206. For example, actuators 206 may extend proximally from compression device 204 to and through actuation device 208 via through holes 304. Through holes 304 may be generally cylindrical, and may be sized such that actuators 206 may move (e.g., distally) through flange 302 via through holes 304. As shown, first end 240 of each of actuators 206 may be connected to a proximal surface of compression device 204 using the same or similar connection mechanisms described above with reference to FIGS. 2A-2D. However, in other examples, first end 240 of actuators 206 may be coupled to other surfaces of compression device 204 (e.g., interior, exterior, or distal surfaces). Second end 242 of each of actuators 206 may be connected to actuation device 208 using the same or similar connection mechanisms described above with reference to FIGS. 2A-2D.

In some examples, at least a portion of each of actuators 206 may be enclosed within a corresponding one of outer tubes 306. Outer tubes 306 may extend proximally from flange 302 to actuation device 208. For example, outer tubes 306 may contact a proximal surface of flange 302 at a location surrounding through holes 304. In some examples, outer tubes 306 may be fixedly coupled to the proximal surface of flange 302. A diameter (e.g., outer diameter) of outer tubes 306 may be larger than a diameter of through holes 304 to prevent outer tubes 306 from extending distally past the proximal surface of flange 302. Such positioning of outer tubes 306 enables outer tubes 306 to apply and maintain pressure distally on flange 302 as actuators 206 are actuated to prevent distal end 118 of shaft 114 of medical device 104 from bending.

Outer tubes 306 and actuators 206 enclosed therein, may extend proximally from flange 302 to actuation device 208 along an exterior (e.g., a radial exterior) of shaft 114 of medical device 104 (e.g., to a proximal end of medical device 104). Actuation device 208 may be mounted to handle 112 at proximal end of medical device 104 (FIG. 1A). In other examples, actuation device 208 may be separately held from medical device 104 and include a handle to allow an operator to grip actuation device 208, as discussed above. Optionally, a proximal portion of outer tubes 306 having actuators 206 enclosed therein may be further enclosed within a conduit that connects to actuation device 208. The conduit may help to prevent outer tubes 306 from inadvertently intertwining or tangling prior to being received by or otherwise coupled to actuation device 208. Additionally or alternatively, at least a portion of the radial exterior of shaft 114 of medical device 104 may include one or more grooves or channels to movably receive portions of outer tubes 306 having actuators 206 enclosed therein.

The second configuration of actuators 206 relative to compression device 204 and actuation device 208 in delivery device 300 results in compression device 204 being moved proximally from distal end 214 to proximal end 212 of assembly 202. FIGS. 3B-3D show three exemplary positions of compression device 204 prior to and as compression device 204 is moved proximally from distal end 214 to proximal end 212 of assembly 202.

In a first position prior to actuation of actuators 206 shown in FIG. 3B (also shown in FIG. 3A), compression device 204 may be configured to surround assembly 202 at distal end 214. For example, the fixed inner diameter of compression device 204 may be greater than an outer diameter of assembly 202 at distal end 214. In some examples, compression device 204 may be sized to fit securely around assembly 202 at distal end 214 such that compression device 204 maintains the first position until actuators 206 are actuated. Additionally or alternatively, although not shown, a fastening or securement mechanism, such as an outwardly protruding wall at distal end 214 of assembly 202 and a corresponding notch on interior surface of compression device 204, may help to secure compression device 204 in the first position until actuators 206 are actuated. When compression device 204 is in the first position, outer wall 226 of assembly 202 may be in the first position having the substantially convex configuration.

After a hemostatic device has been deployed or otherwise positioned at a treatment site as described in detail above with reference to FIGS. 1A-1C, a distalmost end of first set and second set of protrusions 236, 238 of assembly 202 may be placed in contact with or adjacent to the hemostatic device. Then, the operator may actuate (e.g., by rotating, sliding, pushing, and/or pulling) actuation mechanism 244 on actuation device 208 to cause delivery of the deliverable substance. For example, actuators 206 may be pulled or urged proximally causing compression device 204 to move proximally from the first position to a second position, as shown in FIG. 3C. Actuation mechanism 244 may apply tension on actuators 206, while outer tubes 306 maintain a constant pressure against assembly 202.

In the second position, compression device 204 may be between distal end 214 and proximal end 212, and surrounds portion 227 of outer wall 226 of assembly 202 having an outer diameter in the substantially convex configuration that is greater than the fixed inner diameter of compression device 204. Due to the difference in diameters and flexibility of outer wall 226, as compression device 204 is moved toward the second positon, compression device 204 applies force to outer wall 226 causing outer wall 226 to be pushed inward. As a result, outer wall 226 transitions from the first position having the substantially convex configuration, as shown in FIG. 3B, to the second position having the substantially straight configuration, as shown in FIG. 3C. The transition of outer wall 226 to the second position compresses cavity 232 (e.g., the volume of space within cavity 232 decreases), causing the deliverable substance contained therein to become increasingly pressurized, and flow distally from cavity 232 via openings 230.

Delivery of the deliverable substance from each of openings 230 enables the deliverable substance to be distributed over or contemporaneously delivered to a larger surface area of the hemostatic device or treatment site, for example, as compared to conventional injection-based delivery that is limited to application at a single point or location. The distributed delivery may be further enhanced by the vertical space between openings 230 and hemostatic device created by first and second set of protrusions 236, 238.

As the deliverable substance is being delivered, actuators 206 may continue to be pulled proximally, causing compression device 204 to move from the second position to a third position, as shown in FIG. 3D. In the third position, compression device 204 is at proximal end 212 of assembly 202, and surrounds a portion of outer wall 226 of assembly 202 having an outer diameter less than the fixed inner diameter of compression device 204. In some examples, compression device 204 may be sized to fit securely around assembly 202 at proximal end 212 (similar to at distal end 214) such that compression device 204 helps to maintain the third position and cannot be moved any further proximally to prevent compression device 204 from disconnecting from assembly 202. Additionally or alternatively, although not shown, a fastening or securement mechanism, such as an outwardly protruding wall at proximal end 212 of assembly 202 and a corresponding notch on interior surface of compression device 204, may help to secure compression device 204 in the third position. Further, and as shown, an outer diameter of flange 302 may be configured to extend to at least an outer diameter of compression device 204, which prevents any further proximal movement of compression device 204.

As compression device 204 moves from the second position to the third position, and passes portion 227 of outer wall 226 having the outer diameter in the substantially convex configuration greater than the fixed inner diameter of compression device 204, the force applied by compression device 204 is at least partially released. As a result, outer wall 226 may at least partially transition back from the second position having the substantially straight configuration, shown in FIG. 3C, to the first position having the substantially convex configuration, shown in FIG. 3D. In some examples, outer wall 226 may have resilient and/or shape memory properties, which may further help to retain compression device 204 in the third position.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A delivery device comprising:
a tube having a proximal end, a distal end, and a lumen extending from the proximal end to the distal end, wherein the proximal end of the tube is mountable to a distal tip of a medical device via the lumen, and the tube includes:
an inner wall defining the lumen,
an outer wall coupled to the inner wall at the proximal end and the distal end;
a cavity formed from the coupled inner wall and outer wall, the cavity configured to hold a deliverable substance; and
a plurality of openings connected to the cavity and positioned on a distal-facing surface of the distal end,
wherein at least a portion of the inner wall or the outer wall is a flexible wall that transitions between a first configuration and a second configuration to cause delivery of the deliverable substance from the cavity to a treatment site through the plurality of openings when a force is applied to the flexible wall.

2. The delivery device of claim 1, wherein the tube further comprises a plurality of protrusions extending distally from the distal-facing surface.

3. The delivery device of claim 2, wherein the plurality of protrusions are positioned along one or both of an outer circumference of the distal-facing surface and an inner circumference of the distal-facing surface.

4. The delivery device of claim 1, wherein the tube further comprises a plurality of channels connecting the plurality of openings to the cavity.

5. The delivery device of claim 4, wherein each of the plurality of channels includes a proximal end connected to the cavity and a distal end connected to a respective one of the plurality of openings, and wherein each of the plurality of channels is a tapered channel that gradually narrows from the proximal end toward the distal end.

6. The delivery device of claim 1, wherein each of the plurality of openings are configured to form a seal, and wherein the seal breaks when the force is applied to the flexible wall.

7. The delivery device of claim 1, wherein a configuration of the lumen prevents obstruction of components at the distal tip of the medical device when the tube is mounted to the medical device, the components including at least one of distal openings of one or more working channels, one or more illumination devices, or one or more visualization devices.

8. The delivery device of claim 1, wherein at least the portion of the inner wall is the flexible wall, and the force applied to the flexible wall to cause the delivery of the deliverable substance is an extension of the distal tip of the medical device through the lumen from the proximal end to the distal end.

9. The delivery device of claim 8, wherein at least the portion of the inner wall transitions between a concave configuration and a straight configuration, and wherein, in the concave configuration, an innermost diameter of at least the portion of the inner wall is less than an outer diameter of the distal tip of the medical device, causing at least the portion of the inner wall to transition from the concave configuration to the straight configuration when the distal tip of the medical device is extended through the lumen.

10. The delivery device of claim 1, wherein at least the portion of the outer wall is the flexible wall, and the delivery device further comprises:
a compression device surrounding a portion of the tube and configured to move from a first position to a second position along the tube, wherein the force applied to the flexible wall to cause the delivery of the deliverable substance is a movement of the compression device from the first position to the second position along the tube.

11. The delivery device of claim 10, wherein at least the portion of the outer wall transitions between a convex configuration and a straight configuration, and wherein, in the convex configuration, an outermost diameter of at least the portion of the outer wall is greater than an inner diameter of the compression device, causing at least the portion of the outer wall to transition from the convex configuration to the straight configuration as the compression device is moved from the first position to the second position.

12. The delivery device of claim 10, further comprising:
an actuation device; and
one or more actuators connected to the compression device and extended to the actuation device, wherein actuation of the one or more actuators via the actuation device moves the compression device from the first position to the second position.

13. The delivery device of claim 12, wherein the one or more actuators extend distally from the compression device, loop around the distal end of the tube into the lumen, and proceed proximally through a working channel of the medical device to extend to the actuation device.

14. The delivery device of claim 12, further comprising:
a flange attached to the proximal end of the tube and including one or more through holes, wherein the one or more actuators extend from the compression device via the one or more through holes to the actuation device; and
one or more outer tubes coupled to the flange and enclosing the one or more actuators from a proximal surface of the flange to the actuation device.

15. The delivery device of claim 1, wherein:
the tube is formed from an assembly comprised of at least a first assembly component and a second assembly component,
the first assembly component includes the inner wall and a proximal-facing surface of the proximal end, and
the second assembly component includes the outer wall and the distal-facing surface of the distal end.

16. A delivery device comprising:
a lumen extending from a proximal end to a distal end of the delivery device, wherein the proximal end is configured to be mounted to a distal tip of a medical device via the lumen;
an inner wall defining the lumen, wherein at least a portion of the inner wall is a flexible wall having a concave configuration in a first position;
an outer wall coupled to the inner wall at the proximal end and the distal end;
a cavity formed from the coupled inner wall and outer wall, the cavity configured to hold a deliverable substance; and
a plurality of openings connected to the cavity and positioned on a distal-facing surface of the distal end,
wherein when the delivery device is mounted to the distal tip of the medical device and the distal tip of the medical device is extended distally from the proximal end to the distal end through the lumen, at least the portion of the inner wall transitions from the first position to a second position having a substantially straight configuration, the transition compressing the cavity and causing delivery of the deliverable substance from the cavity to a treatment site through the plurality of openings.

17. The delivery device of claim 16, wherein the delivery device further comprises a plurality of protrusions distally extending from the distal-facing surface, the plurality of protrusions positioned along at least one of an outer circumference or an inner circumference of the distal-facing surface.

18. A delivery device comprising:
an assembly forming a tube having a proximal end and a distal end that is mountable to a medical device, the assembly including:
a first assembly component including an inner wall of the tube and a proximal-facing surface of the proximal end;
a second assembly component coupled to the first assembly component at the proximal end and the distal end, the second assembly component including an outer wall of the tube and a distal-facing surface of the distal end, wherein at least a portion of the outer wall is a flexible wall transitionable between a convex configuration and a substantially straight configuration, and wherein the distal-facing surface includes a plurality of openings; and
a cavity formed from the coupled first assembly component and the second assembly component, the cavity connected to the plurality of openings and configured to hold a deliverable substance;
a compression device surrounding the assembly and movable from a first position to a second position along a radial exterior of the assembly; and
one or more actuators connected to the compression device and configured to move the compression device from the first position to the second position,
wherein when the one or more actuators are actuated, at least the portion of the outer wall transitions from the convex configuration to the substantially straight configuration as the compression device is moved from the first position to the second position, the transition compressing the cavity and causing delivery of the deliverable substance from the cavity to a treatment site through the plurality of openings.

19. The delivery device of claim 18, further comprising:
an actuation device connected to a proximal end of the one or more actuators, wherein the one or more actuators extend distally from the compression device, loop around the distal end of the tube into a lumen defined by the inner wall of the tube, and proceed proximally through a working channel of the medical device to extend to the actuation device.

20. The delivery device of claim 18, further comprising:
a flange connected to the proximal end of the assembly, the flange having one or more through holes; and
an actuation device connected to a proximal end of the one or more actuators, wherein the one or more actuators extend from the compression device via the one or more through holes to the actuation device.
